(19) United States
(12) Patent Application Publication
(10) Pub. No.: US 2024/0139395 A1
(43) Pub. Date: May 2, 2024

(54) FILTER ELEMENT AND METHOD FOR ITS PRODUCTION

(71) Applicant: Sefar AG, Heiden (CH)

(72) Inventor: Hansjörg DÜR, Schwarzach (AT)

(73) Assignee: Sefar AG, Heiden (CH)

(21) Appl. No.: 18/280,976

(22) PCT Filed: May 23, 2022
(86) PCT No.: PCT/EP2022/063896

§ 371 Date: Sep. 8, 2023

(30) Foreign Application Priority Data
Jun. 2, 2021 (EP) 21177303.1

(51) Int. Cl. A61M 1/36

(52) U.S. Cl. CPC A61M 1/3635; A61M 2202/0028; A61M 2202/0413; A61M 2205/02; A61M 2207/00

(57) ABSTRACT

The invention relates to a filter element which is configured in the form of a pocket or a tube with an inner space extending in a longitudinal direction and has a multilayer structure which has a permeable outer layer, a permeable inner layer and at least one intermediate layer arranged between the outer layer and the inner layer, which is configured as a fine-pored filter element, wherein the filter element has a predetermined axial length, and wherein the outer layer and the inner layer have the predetermined axial length. It is according to the invention that the intermediate layer is formed axially shorter than the outer layer and the inner layer to form an overflow region disposed at an axial upper end portion of the filter element, wherein an upper edge of the intermediate layer is spaced axially downward from the upper edge of the outer layer and the inner layer, that the outer layer, the inner layer and the intermediate layer are firmly connected to one another along at least one longitudinal edge to form a longitudinal connecting seam, and that the outer layer and the inner layer are firmly connected to one another along their upper edge to form an upper transverse connecting seam, wherein the upper edge of the intermediate layer is arranged free and unconnected between the outer layer and the inner layer.

The invention relates to a filter element, in particular a blood filter element, which is configured in the form of a pocket or tube with an inner space extending in a longitudinal direction and has a multilayer structure which has a permeable outer layer, a permeable inner layer and at least one intermediate layer arranged between the outer layer and the inner layer, which is configured as a fine-pored filter element, wherein the filter element has a predetermined axial length, and wherein the outer layer and the inner layer have the predetermined axial length according to the preamble of Claim 1.

The invention further relates to a method for producing such a filter element, in particular such a blood filter element, according to Claim 13.

Filter elements of this type, which are used as blood filters, are used in particular in so-called heart-lung machines to purify circulating blood and to retain excessively large gas bubbles in the blood.

WO 98/15302 A1 discloses a blood filter element with three layers, wherein the individual layers are attached to a rigid base body or frame. The frame predefines a cylindrical shape.

DE 2 530 413 C3 discloses a bag-shaped blood filter formed by welding together different layers.

The object underlying the invention is to provide a simple filter element having a robust structure, in particular a blood filter element, and a method for its production, with which a particularly efficient filtration, in particular of blood, can be carried out.

The object is achieved on the one hand by a filter element having the features of claim 1 and on the other hand by a method having the features of claim 13. Preferred embodiments of the invention are indicated in the dependent claims.

The filter element according to the invention is characterized in that the intermediate layer is formed axially shorter than the outer layer and the inner layer along with the formation of an overflow region, which is arranged in an axial upper end portion of the filter element, wherein an upper edge of the intermediate layer is axially spaced towards the bottom from the upper edges of the outer layer and the inner layer, in that the outer layer, the inner layer and the intermediate layer are firmly connected to one another along at least one longitudinal edge along with the formation of a longitudinal connecting seam, and in that the outer layer and the inner layer are firmly connected to one another along their upper edge along with the formation of an upper transverse connecting seam, wherein the upper edge of the intermediate layer is arranged free and unconnected between the outer layer and the inner layer.

A basic idea of the invention is that, in the case of a multilayer structure of a sock- or tube-shaped filter element, the filter element is configured as an intermediate layer between an outer layer and an inner layer, but with a different length in relation thereto. The intermediate layer is configured to be axially shorter in a defined manner, so that an upper edge of the intermediate layer is spaced apart from the upper edges of the outer layer and the inner layer along with forming a free space. In the upper section of the filter element, there is thus only a two-layer structure, whereby an overflow region is formed. The overflow region without the intermediate layer has a lower flow resistance. Thus, in certain situations, excess fluid can be discharged quickly and efficiently via the overflow region, while the outer layer and the inner layer ensure a minimum filtering effect.

Another aspect of the invention is that it is sufficient for a positional fixation of the intermediate layer in the filter element if there is a firm connection with the outer layer and the inner layer at least along a longitudinal edge with a longitudinal connecting seam. In particular, the invention is based on the knowledge that an upper edge of the intermediate layer can remain free and unconnected. In particular, the use of additional fusible foils for connecting internal layers can thus be dispensed with during the production of the filter element. A sufficiently robust structure is achieved by connecting the intermediate layer along a longitudinal edge to the other two layers and by connecting the outer layer and the inner layer at the upper edge. A large and effective filtering surface is achieved by a small number of connecting seams.

Thus, with efficient production, a particularly advantageous and functional structure of a filter element can be achieved.

The filter element according to the invention is basically installed in such a way that the fluid to be purified, in particular blood, flows into the inner space from above or from the front. For purifying, the fluid passes radially to the outside through the approximately hollow-cylindrical peripheral wall of the filter element. The filter element can be tubular with an upper and a lower opening or alternatively have a pocket or sock shape in which a lower opening is closed. In this case, the filter element is operated as a whole in such a way that, in normal filter operation, the fluid to be purified, in particular blood, passes through the wall of the filter element through the lower and middle regions, which are formed with three layers. An additional filtering effect, in particular a cascade filtration together with the intermediate layer, can be achieved by a corresponding design of the inner layer. If an unexpected clogging or foaming of the fluid occurs in this area, an overflow of the fluid can also take place radially outwards via the two-layer overflow region. This ensures sufficient blood circulation, for example through a heart-lung machine, and thus for the patient, particularly when the filter element is used as a blood filter element.

In principle, the fluid to be purified, such as blood, can also flow radially from the outside to the inside in an alternative embodiment.

A preferred embodiment of the invention is that the outer layer, the inner layer and/or the intermediate layer are firmly connected to one another at their lower edge along with the formation of a lower transverse connecting seam. In each case, only two layers or all layers can be connected to one another in an annular manner. This increases the stability of the structure. For a pocket-shaped filter element, the lower transverse connecting seam is formed in such a way that an opening of the inner space is completely closed towards the bottom.

In general, the filter element according to the invention can be constructed in such a way that a starting material with the three layers arranged one above the other, which are assembled according to the desired length and a double width, is folded once and folded over one another. To form the filter element, which is in particular tubular in shape, a fixed connection can then be formed along the longitudinal edge, so that a filter element is formed with only one longitudinal connecting seam. The tube shape can be cylindrical or conical.

A particularly stable configuration of the invention is that a structure of two halves is provided and that the two halves are firmly connected to one another along the two longitudinal edges along with the formation of two longitudinal connecting seams. The respective halves can be assembled in the same way and each have the three layers. In accordance with the invention, the intermediate layer is defined as being axially shorter than the outer layer and the inner layer.

Another preferred embodiment of the invention is that an axial length of the overflow region is between 5% to 30% of the axial length of the filter element. In the actual filter area, a side wall of the filter element is formed of three layers, while in the overflow region a two-layer structure is formed of the outer layer and the inner layer without an intermediate layer.

In principle, the layers can be connected by any suitable connecting method. It is particularly advantageous in terms of production technology that at least one connecting seam is formed by ultrasonic welding. This allows in particular polymeric materials having narrow connecting width, to be firmly interconnected efficiently and reliably with a narrow and proper connecting seam.

For forming a pocket-shaped filter element, in particular a blood filter element, it is provided according to one variant of the invention that the lower transverse connecting seam is configured to form a closed bottom. This prevents free passage of fluid, such as blood, towards the bottom and out of the filter element. Thus, purifying of the entire fluid entering the filter element can be ensured.

In general, the fine-pored intermediate layer for filtering can be formed in any suitable manner. According to a further development of the invention, it is particularly expedient for the filter element to comprise a filter woven fabric, a filter membrane, a filter knitted fabric and/or a non-woven filter fabric. According to the intended filtration task, a pore or aperture size, an aperture proportion of the filtering surface, and a filtering capacity or a flow resistance are to be selected.

According to a further development of the invention, it is particularly advantageous that an aperture or pore size of the filter element is between 10 μm and 400 μm, in particular between 25 μm and 60 μm. This can be achieved in particular by configuring the filter element as a filter woven fabric, wherein preferably an aperture proportion of between 30% and 60% of the total filtering surface can be reached. This is possible in particular by using very fine filaments, which are preferably of between 10 μm to 30 μm, particularly preferably between 18 μm to 28 μm.

In principle, the outer layer and the inner layer have a permeable structure, in particular with a larger aperture or pore size than the intermediate layer. According to one variant of the invention, it is advantageous that the outer layer and/or the inner layer is formed as a woven fabric, a knitted fabric, a net, a grate and/or a nonwoven fabric. The use of a woven fabric or knitted fabric, which is preferably formed with monofilament or multifilament threads, is particularly appropriate. This largely prevents parts from becoming detached from the layer.

A particularly good purification effect is achieved by the fact that the material of the intermediate layer, the outer layer and the inner layer is a polymeric material, in particular polyester or polyamide. The material is a medically approved, particularly pure polymer material.

According to a further development of the invention, it is also particularly expedient for the material of the intermediate layer, the outer layer and the inner layer to be the same. The use of the same material improves applicability in the medical sector and also facilitates approval as a medical product. Particularly in combination with welding, especially ultrasonic welding, a mono-material filter element can be formed which is free of additional adhesives or other materials. This can ensure a particularly high level of product safety.

A further advantageous embodiment of the invention is that an aperture size of the outer layer and/or the inner layer is between 100 μm and 400 μm, in particular between 150 μm and 300 μm. As a result, a flow resistance in the filter element is hardly affected with respect to the intermediate layer performing the relevant filtering. In addition, a good overflow function for the overflow region is achieved with this size range, particularly with an aperture proportion of more than 50% in the outer layer and the inner layer.

With regard to the method for producing a filter element, in particular a blood filter element, the invention is characterized in that the outer layer and the inner layer are firmly connected to one another at their upper edges to form an upper transverse connecting seam, wherein the intermediate layer is arranged in an intermediate space between the outer layer and the inner layer and an upper edge of the intermediate layer remains free and unattached. In doing so, a filter element having an overflow function in an upper region is achieved by an only two-layer structure, with an upper edge of the filtering intermediate layer remaining free and unattached between the outer layer and the inner layer.

The method can be used to efficiently produce, in particular, the filter element described above, such as a blood filter element, while achieving the advantages described above.

According to a further development of the invention, a particularly good connection between the individual flexible layers can be provided by connecting the outer layer, the inner layer and/or the intermediate layer at their outer edges by ultrasonic welding, thermal welding, gluing and/or hot bonding. In particular, ultrasonic welding and thermal welding can eliminate the need for adhesives or additional components.

According to a further method variant of the invention, it is expedient that a pocket- or tube-shaped intermediate shape with outer connecting seams is formed by connecting the flexible layers, and that the intermediate shape is turned inside out to form the filter element, with the connecting seams being turned inwards. In this way, a protected arrangement of the connecting seams on the intermediate product can be achieved. In the case of a flow to the filter element from the inside, it is also ensured that even if the smallest components detach from the connecting seam, they cannot enter the actual blood circuit when used as a blood filter element, since they are then caught by the filtering intermediate layer downstream in the direction of flow.

As already mentioned, the filter element according to the invention can preferably be used as a blood filter element, for example in a heart-lung machine. However, it is also suitable for filtering other fluids, which according to the invention can be considered in particular as liquids with or without solids and with or without gaseous components.

The invention is further described below with reference to preferred embodiments, which are shown schematically in the drawings. The drawings show in:

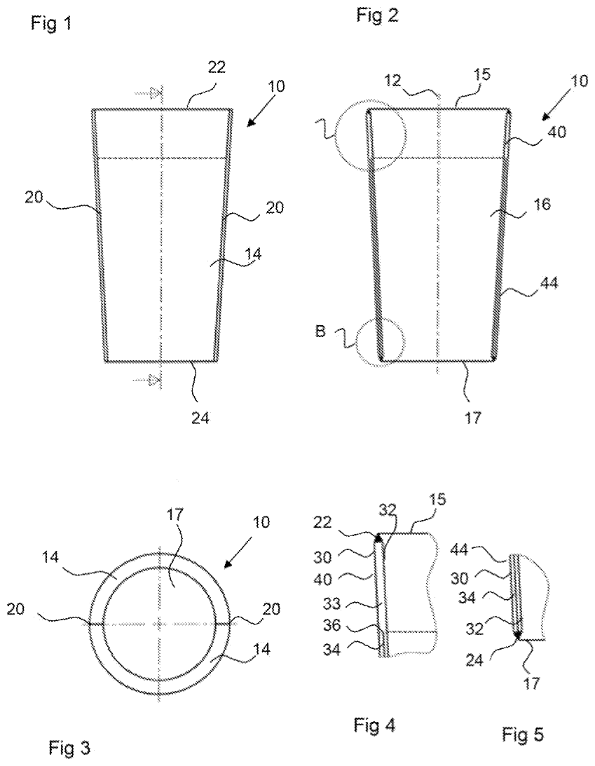

FIG. 1 a side view of a first filter element according to the invention;

FIG. 2 a cross-sectional view of the filter element of FIG. 1;

FIG. 3 a top view of the filter element according to FIGS. 1 and 2;

FIG. 4 an enlarged illustration of detail A of FIG. 2;

FIG. 5 an enlarged illustration of detail B of FIG. 2;

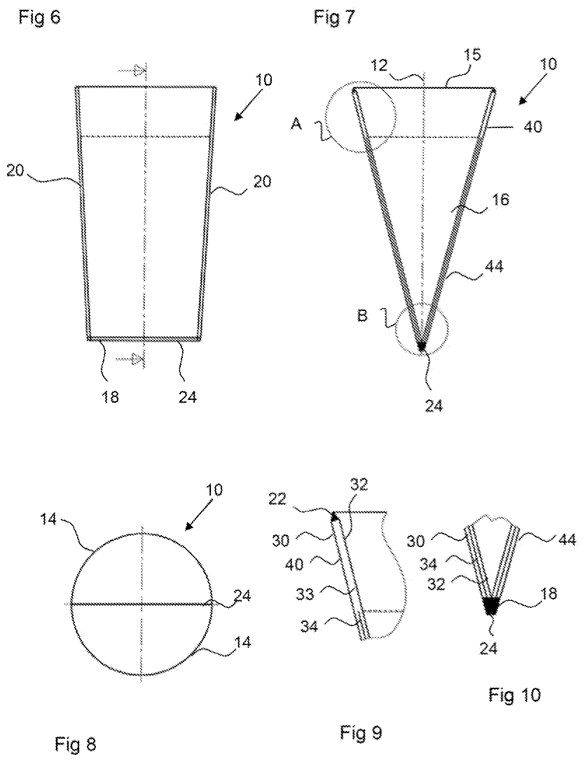

FIG. 6 a side view of a second filter element according to the invention;

FIG. 7 a cross-sectional view of the filter element of FIG. 6;

FIG. 8 a top view of the filter element according to FIGS. 6 and 7;

FIG. 9 an enlarged illustration of detail A of FIG. 7; and

FIG. 10 an enlarged illustration of detail B of FIG. 7;

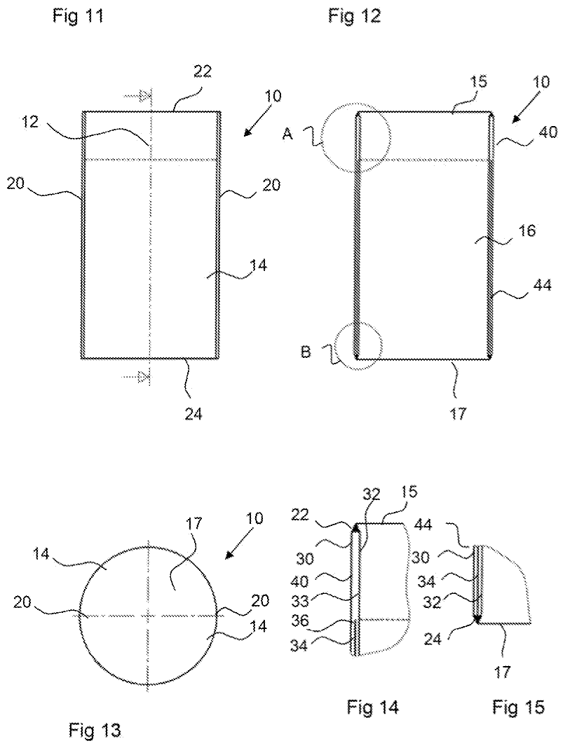

FIG. 11 a side view of a third filter element according to the invention;

FIG. 12 a cross-sectional view of the filter element of FIG. 11;

FIG. 13 a top view of the filter element according to FIGS. 11 and 12;

FIG. 14 an enlarged illustration of detail A of FIG. 12; and

FIG. 15 an enlarged illustration of detail B of FIG. 12;

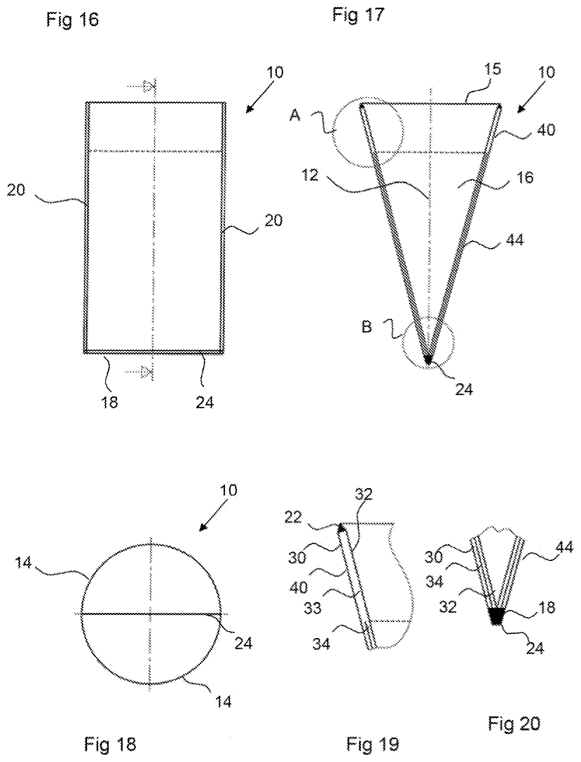

FIG. 16 a side view of a fourth filter element according to the invention;

FIG. 17 a cross-sectional view of the filter element of FIG. 16;

FIG. 18 a top view of the filter element according to FIGS. 16 and 17;

FIG. 19 an enlarged illustration of detail A of FIG. 17; and

FIG. 20 an enlarged illustration of detail B of FIG. 17.

FIGS. 1 to 5 illustrate a first exemplary embodiment of a filter element 10 according to the invention, which is tubular with a continuous inner space 16 having an upper opening 15 and a lower opening 17. The tubular filter element 10 is slightly conical along a longitudinal axis 12 towards the lower opening 17. The openings 15, 17 are approximately circular in shape.

The filter element 10 is formed from two halves 14, which are connected to one another along two longitudinal edges along the longitudinal axis 12 with longitudinal connecting seams 20. The connecting may be performed, in particular, by means of ultrasonic welding. Each half 14 has three layers, namely an outer layer 30, an inner layer 32 and an intermediate layer 34 arranged therebetween in an intermediate space 33. The intermediate layer 34 constitutes a fine-pored filter element, while the outer layer 30 and the inner layer 32 may have larger pore apertures. The outer layer 30 and the inner layer 32 have the same axial length, while the intermediate layer 34 is axially shorter in a defined manner.

In the filter element 10 shown, the three layers are connected to one another along the two longitudinal connecting seams 20 and along a lower transverse connecting seam 24, for example by ultrasonic welding. The lower transverse connecting seam 24 is of annular design and thus encloses the lower opening 17. The region having the total of three layers forms a filter area 44, which extends over approximately 80% of the axial length of the filter element 10 from bottom to top.

In an upper region, a wall of the filter element 10 is formed only of two layers with the outer layer 30 and the inner layer 32, as is shown illustratively in FIG. 4. The outer layer 30 and the inner layer 32 are welded together via an annular-design upper transverse connecting seam 22, with the intermediate space 33 being closed off. This two-layer section forms an overflow region 40 in the filter element 10, which allows improved flow of fluid through the wall in the event of an upward fluid backpressure due to the significantly lower flow resistance.

At the lower end of the overflow region 40, the intermediate layer 34 ends, wherein an upper edge 36 of the intermediate layer 34 comes to lie freely in the intermediate space 33 between the outer layer 30 and the inner layer 32, without a direct fixed connection of the upper edge 36 to the adjacent outer layer 30 or the adjacent inner layer 32.

Referring to FIG. 5, to terminate the lower filter area 44, the outer layer 30, the inner layer 32 and the intermediate layer 34 are welded together and firmly connected via the annular-design lower transverse connecting seam 24.

In the exemplary embodiment shown, the fluid to be purified flows through the upper opening 15 along the longitudinal axis 12. Due to an applied pressure difference, the fluid can flow radially outward from an inner space 16 of the filter element 10. In a normal fill ratio, this occurs through the three-layer filter area 44, while in the event of a possible back pressure, radial flow can also occur in the upper overflow region 40. When used as a blood filter element, the pore structure of the outer layer 30 and the inner layer 32 can be configured in such a way that larger particle sizes and gas bubbles that are critical for a patient are retained, even if the same filtering and purification effect is not achieved as with flow through the filter area 44 with the additional intermediate layer 34.

FIGS. 6 to 10A show a further possible embodiment of a filter element 10 according to the invention. This filter element 10 has basically the same structure as the above-described filter element 10 according to FIGS. 1 to 5, but whereby a pocket-like structure with a closed bottom 18 is formed. In this case, the filter element 10 is also made from two halves 14 by superimposing and welding along the longitudinal edges with longitudinal connecting seams 20. In addition, the outer layer 30, the inner layer 32 and the intermediate layer 34 disposed therebetween are also firmly connected to one another at their lower ends by a linear, lower transverse connecting seam 24, thereby forming a closed bottom 18.

In this case, the intermediate layer 34, which comprises the actual fine-pored filter element, extends from the lower end as far as to about 80% of the total length of the filter element 10, forming a three-layer filter area 44. An overflow region 40 is formed in the remaining two-layer upper area with the outer layer 30 and the inner layer 32, which allows an easier flow through the wall of the filter element 10.

Also in this exemplary embodiment of a filter element 10, an inflow of the fluid to be purified takes place from above via a substantially circular opening 15 into an inner space 16 of the filter element 10. Due to an applied pressure difference, the fluid can pass from the inside to the outside of the wall either of the three-layer filter area 44 or, in the case of a corresponding filling level, radially outward through the two-layer overflow region 40.

At the upper end, the outer layer 30 and the inner layer 32 are directly welded together via an upper transverse connecting seam 22, which is of annular design in order to form the upper opening. Overall a sock-like or tent-like structure of the filter element 10 is thus achieved.

FIGS. 11 to 15 illustrate a third embodiment of a filter element 10 according to the invention. This filter element 10 is substantially the same as the filter element 10 according to the first embodiment shown in FIGS. 1 to 5. It differs from this first embodiment only in that, as can be seen by comparing FIGS. 1 and 2 with FIGS. 11 and 12, the filter element 10 does not taper towards the bottom. Accordingly, it has a substantially cylinder-like shape.

FIGS. 16 to 20 illustrate a fourth embodiment of a filter element 10 according to the invention. This filter element 10 is substantially the same as the second embodiment, also having a pocket-like structure with a closed bottom 18. In contrast to the second exemplary embodiment, however, this embodiment extends conically towards the lower opening 17 in only one dimension. This can be seen in particular by comparing FIGS. 6 and 16.

1. A filter element, in particular blood filter element, which is configured in the form of a pocket or tube with an inner space extending in a longitudinal direction and has a multilayer structure which comprises a permeable outer layer, a permeable inner layer and at least one intermediate layer (34) which is arranged between the outer layer and the inner layer and is configured as a fine-pored filter element, wherein the filter element has a predetermined axial length and the outer layer and the inner layer have the predetermined axial length,
wherein
the intermediate layer is formed axially shorter than the outer layer and the inner layer along with the formation of an overflow region which is arranged at an axial upper end area of the filter element, wherein an upper edge of the intermediate layer is axially spaced downwards from the upper edge of the outer layer and the inner layer,
the outer layer, the inner layer and the intermediate layer are firmly connected to one another along at least one longitudinal edge along with the formation of a longitudinal connecting seam, and
the outer layer and the inner layer are firmly connected to one another along their upper edge along with the formation of an upper transverse connecting seam, wherein the upper edge of the intermediate layer is arranged free and unconnected between the outer layer and the inner layer.

2. The filter element according to claim 1,
wherein
the outer layer, the inner layer and/or the intermediate layer are firmly connected to one another at their lower edge along with the formation of a lower transverse connecting seam.

3. The filter element according to claim 1,
wherein
a structure composed of two halves is provided and
the two halves are firmly connected to one another along the two longitudinal edges along with the formation of two longitudinal connecting seams.

4. The filter element according to claim 1,
wherein
an axial length of the overflow region is between 5% to 30% of the axial length of the filter element.

5. The filter element according to claim 1,
wherein
at least one connecting seam is formed by ultrasonic welding.

6. The filter element according to claim 2,
wherein
the lower transverse connecting seam is configured to form a closed bottom.

7. The filter element according to claim 1,
wherein
the filter element comprises a filter woven fabric, a filter membrane, a filter knitted fabric and/or a non-woven filter fabric.

8. The filter element according to claim 1,
wherein
an aperture or pore size of the filter element is between 10 μm and 400 μm, in particular between 25 and 60 μm.

9. The filter element according to claim 1,
wherein
the outer layer and/or the inner layer is formed as a woven fabric, knitted fabric, net, grate and/or nonwoven fabric.

10. The filter element according to claim 1,
wherein
the material of the intermediate layer, the outer layer and the inner layer is a polymeric material, in particular polyester or polyamide.

11. The filter element according to claim 1,
wherein
the material of the intermediate layer, the outer layer and the inner layer is the same.

12. The filter element according to claim 1,
wherein
an aperture size of the outer layer and/or the inner layer is between 100 μm and 400 μm, in particular between 150 μm and 300 μm.

13. The method for producing a filter element, in particular a blood filter element, according to claim 1,
wherein
the outer layer and the inner layer are firmly connected to one another at their upper edges along with the formation of an upper transverse connecting seam, wherein the intermediate layer is arranged in an intermediate space between the outer layer and the inner layer, and an upper edge of the intermediate layer remains free and unattached.

14. The method according to claim 13,
wherein
the outer layer, the inner layer and/or the intermediate layer are connected at their outer edges by ultrasonic welding, thermal welding, adhesive bonding and/or hot bonding.

15. The method according to claim 13,
wherein
by connecting the layers a pocket- or tube-shaped intermediate shape with external connecting seams is formed, and
in that the intermediate shape is turned inside out to form the filter element, wherein the connecting seams are turned inwards.
